(19) United States
(12) Patent Application Publication
(10) Pub. No.: US 2004/0170691 A1
(43) Pub. Date: Sep. 2, 2004

(54) Amoxycillin pellets

(76) Inventor: Francis Walter Grimmett, Rustington (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/450,697

(22) PCT Filed: Dec. 20, 2001
(86) PCT No.: PCT/GB01/05682

(30) Foreign Application Priority Data
Dec. 21, 2000 (GB) 00312678

(51) Int. Cl. A61K 31/43; A61K 9/14

(52) U.S. Cl. 424/489; 514/192

(57) ABSTRACT

Pelletised amoxycillin trihydrate for improved processability.

The present invention relates to a pelletised form of amoxycillin trihydrate, in particular for formulation in combination with potassium clavulanate.

Amoxycillin trihydrate is a well known anti-bacterial agent is available widely in a number of different presentations, for instance, capsules, tablets, dry powders/granules for reconstitution into aqueous suspension, and i.v. formulations.

In addition, formulations are also available comprising amoxycillin trihydrate in combination with the β-lactamase inhibitor potassium clavulanate, for instance those marketed by SmithKline Beecham under the trade mark Augmentin.

There is a need to provide improved forms of amoxycillin trihydrate to assist processing of the material, in particular to assist in its formulation with potassium clavulanate. Potassium clavulanate is recognised to be a highly moisture sensitive compound, for which special precautions such as a low humidity, preferably below 30%, have to be observed during secondary manufacturing operations. Accordingly, controlling the moisture content of the amoxycillin trihydrate used in such formulations also comprising potassium clavulanate is beneficial.

Conventionally, amoxycillin trihydrate has been used as a powdered or directly precipitated material, without further deliberate processing. It has however been unexpectedly found that processability is improved if the amoxycillin trihydrate is pelletised.

Accordingly, in a first aspect, the present invention provides pelletised amoxycillin trihydrate.

Pelletised amoxycillin trihydrate can be dried more efficiently than powdered material, is safer to handle as there is less dust released, and handling is more efficient as the material is easier to transfer both manually and by vacuum. Furthermore, during secondary manufacturing operations, it is found that the pelletised material is easier to mill into granules or into a denser fine powder and that densification can be produced in one rather than two or more steps, as needed with powdered or directly precipitated material.

Pelletised amoxycillin trihydrate comprises pellets of amoxycillin trihydrate which are small cylinders which typically have a diameter in the range 1 to 5 mm, preferably about 2 to 3 mm and a length in the range 1 to 10 mm, preferably about 3 to 4 mm, obtainable by processing a wet cake of amoxycillin trihydrate in a pelletising machine and then drying the thus formed pellets.

In a further aspect, the present invention also provides a process for preparing pelletised amoxycillin trihydrate comprising:

forming a slurry of amoxycillin trihydrate in a suitable solvent;

filtering the slurry to remove solvent and form a wet cake;

optionally washing the wet cake;

forcing this wet cake through a pelletising machine; and thereafter,

drying the pellets thus formed.

Typical solvents for the slurry include aqueous and non-aqueous solvents, for instance water or methyl iso-butyl ketone, or a mixture thereof, preferably water and methyl iso butyl ketone. Typical solvents for washing the wet cake include water followed by methyl iso-butyl ketone.

Typically, the slurry is filtered, preferably through a pressure plate filter, to remove the majority of the process solvent, followed by washing, to form a wet cake, prior to processing in a pelletising machine.

Pelletising machines are well known in the art and include those available from Hosokawa Bepex GmbH.

The initially formed pellets are dried, for instance in a plate drier with warmed plates and in a warm atmosphere of nitrogen, for sufficient time to reduce the moisture content to the desired level, for instance from 10 to 15% water, typically 12 to 14, preferably about 13%.

Furthermore, it is preferred that pelletised amoxycillin trihydrate according to the present invention has an equilibrium relative humidity (ERH) which is less than 40%, preferably less than 30%, more preferably less than 25%, most preferably less than 15%, as this is beneficial for subsequent co-formulation with potassium clavulanate.

The parameter “equilibrium relative humidity” (ERH) refers to the relative humidity that moisture-containing material exhibits when in equilibrium with a particular environment It is an indicator of the amount of “free” moisture in the sample and indirectly of its “dryness” and therefore capability to donate water vapour to materials it may be in contact with. This may be distinguished from the “moisture content” which measures all the water present in the bulk solid, that is chemically bound and free moisture.

The ERH of the amoxycillin trihydrate may be controlled by pre-drying during or after the bulk production process. Methods of drying amoxycillin trihydrate are well known in the art. In a representative method amoxycillin trihydrate is dried on trays in a Krauss Mefei oven, at a temperature in the range 65 to 85° C., preferably about 72° C., in a nitrogen atmosphere maintained at a temperature in the range 65 to 85° C., the temperature being adjusted in response to the progress of the drying operation, as indicated by the ERH of the amoxycillin trihydrate.

The ERH may be measured by conventional methods well known to those skilled in the art, for instance, using commercially available ERH meters, from companies such as Novasina and Humitec.

Preferably, the initial ERH of the amoxycillin trihydrate is as low as 15%, to allow for some increase during subsequent handling.

The pelletised amnoxycillin trihydrate of the present invention may used directly in further processing, for instance blending with a lubricant such as magnesium stearate and then compacting, for instance by slugging or using a roller compactor, to form granules which may then be blended with further lubricant to form a capsule filling mixture.

The pelletised amoxycillin trihydrate of the present invention is especially suited for use in pharmaceutical formulations comprising potassium clavulanate. The pellets for such use may be subjected to a preliminary milling step, before blending with potassium clavulanate (blended with a diluent such as microcrystalline cellulose (for instance, Avicel) or silica gel (for instance, Syloid), to provide particles of similar size.

Accordingly, in a further aspect, the present invention provides a process for the preparing a pharmaceutical formulation comprising amoxycillin trihydrate and potassium clavulanate which comprises mixing together optionally milled, pelletised amoxycillin trihydrate and potassium clavulanate, preferably in the presence of a lubricant such as magnesium stearate and then compacting, for instance by slugging or using a roller compactor.

Preferably, amoxycillin trihydrate and potassium clavulanate are present in a ratio by weight of 1:1 to 20:1. Representative examples include 2:1, 4:1, 7:1, 8:1, 14:1, and 16:1.

Representative formulations include tablets, including swallow tablets, dispersible tablets and chewable tablets, granules, single dose sachets, and dry powders or granules for reconstitution into aqueous syrups. Examples of such formulations are already commercially available from SmithKline Beecham, and are well known (see for instance Physicians Desk Reference, Medical Economics Co, 52 edition, 1998, 2802).

The invention is illustrated by the following examples:

### EXAMPLES

### Example 1

### Pelletisation of Amoxycillin Trihydrate

An aqueous slurry of amoxycillin trihydrate (10% w/w) was fed under pressure at a flow rate of between 600 and 900 litres per hour into a BHS Werke Pressure Plate Filter K-6. The mother liquors were removed prior to a water wash at 240 litres/hr and a methyl iso-butyl ketone wash at 80 litres/hr. The amoxycillin was then blown with nitrogen to remove excessive solvent. The material exited the filter with a consistency of 60% amoxycillin, 29% water and 11% MIBK. The material was gravity fed into a Hosokawa Bepex GmbH GCS 200-80 Pelletiser at a rate of between 90 and 130 kg per hour. The product entered the working zone of the gearwheels, was gripped by the teeth, compressed and forced through holes (3.2 mm in diameter and 6.25 mm deep) into the internal bores of the rollers. The rollers were rotating at a rate of between 47 and 49 rpm. The pelletised material shears off and falls via gravity into the drier. The drier was a Krauss-Maffei GTT-20/8-2.5-2/90 Plate Drier. The pellets passed through the drier across all eight plates. Heat was supplied to the pellets via water at between 68 and 74° C. circulating in the plates and nitrogen at between 72 and 75° C. circulating through the drier. Each pellet had a residence time in the drier of between 50 and 70 minutes. The dried pellet exited from the base of the drier via a rotary valve. The amoxycillin trihydrate pellets had a composition of 86.5% amoxycillin and 13% water.

For further processing, an Apex mill may be used, for instance operating at 7200 rpm, with hammers forward and 0.020 inch screen.

### Example 2

### Using Pellets of Amoxicillin Trihydrate in Secondary Production

Operations to prepare a Capsule Filling Mix

Pellets of amoxycillin trihydrate were broken down, by passing through a grater fitted with a No 2 screen and loaded by vacuum into a suitable blender. The resulting granules were blended with magnesium stearate. This mix was then slugged to a nominal density of 0.375 g/mm, with limits of 0.355 g/mm to 0.395 g/mm. The slugs were milled through an Apex mill, operating typically at 2,900 rpm and fitted with a 0.049 inch screen with knives facing forward. The granules were blended with a further portion of magnesium stearate to convert them into a capsule filling mix with a minimum packed bulk density of 0.80 g/cc.

In comparison, this operation using powdered amoxicillin trihydrate takes two slugging operations, with similar operating conditions, to achieve the required packed bulk density.

### Example 3

### Using pellets of amoxicillin trihydrate in Tablet Production

Pellets of amoxicillin trihydrate were passed through a mill operating at 2,900 rpm, fitted with a 0.040 inch screen with knives facing forward to form milled pellets which were then loaded by vacuum into a suitable blender. Blends of potassium clavulanate with Avicel or Syloid (1:1) plus magnesium stearate were also loaded into the blender. The resulting mix was then slugged to a ratio of 0.390 g/mnn, with limits 0.370 g/mm to 0.410 g/mm. The slugs were passed through an Apex mill, operating at 1000 rpm, fitted with a 0.063 inch screen and with knives facing forward. Other ingredients such as sodium starch glycollate, magnesium stearate and colloidal silicon dioxide (Aerosil) are added to form a compression mixture with a packed bulk density in excess of 0.80 g/cc. Tablets were then obtained by processing this compression mixture in tabletting machine, for instance a Kilian IX 25, a rate which exceeded 70,000 tabs/hr.

In comparison, when using powdered amoxicillin trihydrate, the slugging operation is found to take at least 50% longer due to poorer powder flow. The resultant compression mix is finer, leading to a slower tabletting operation (less than 70,000 tabs/hr).

1. Pelletised amoxycillin trihydrate which has a moisture content of from 10 to 15% water.

2. Pelletised amoxycillin trihydrate as claimed in claim 1 which has an ERH value of less than 40% preferably less than 30%, more preferably less than 25%, most preferably less than 15%.

3. A pharmaceutical formulation comprising pelletised amoxycillin trihydrate as defined in claim 1.

4. A pharmaceutical formulation as claimed in claim 3 further comprising potassium clavulanate.

5. A process for preparing pelletised amoxycillin trihydrate comprising:
forming a slurry of amoxycillin trihydrate in a suitable solvent;
filtering the slurry to remove solvent and form a wet cake;
optionally washing the wet cake;
forcing this wet cake through a pelletising machine; and thereafter, drying the pellets thus formed.
